(19) United States
(12) Patent Application Publication
(10) Pub. No.: US 2018/0328903 A1
(43) Pub. Date: Nov. 15, 2018

(54) MULTI-POWER SOURCE PERISHABLE ITEM SENSOR APPARATUS

(71) Applicant: QUALCOMM Incorporated, San Diego, CA (US)

(72) Inventor: William Henry VON NOVAK, III, San Diego, CA (US)

(21) Appl. No.: 15/593,489

(22) Filed: May 12, 2017

(51) Int. Cl. G01N 33/02; H02J 50/40; H02J 50/10; H02J 50/20; H04B 1/00; G08B 21/02

(52) U.S. Cl. CPC G01N 33/02; H02J 50/40; H02J 50/10; H04L 67/12; H04B 1/0064; G08B 21/02; H02J 50/20

(57) ABSTRACT

Certain aspects of the present disclosure relate to methods and apparatus for implementing a sensor apparatus for a perishable item. The sensor apparatus may include a lower frequency (LF) power receiver that is configured to receive power from a LF wireless transmission, a higher frequency (HF) power receiver that is configured to receive power from a HF wireless transmission, and a LF transmitter configured to transmit sensor data using the power received by the HF power receiver from the HF wireless transmission.

### FIELD

The present disclosure relates generally to sensor and communication systems, and more particularly, to methods and apparatus for implementing a multi-power source perishable item sensor apparatus, such as a microwavable food sensor.

### BACKGROUND

Food safety, shipping, and preparation is a multi-billion dollar endeavor that has developed to help people who have been dealing with food spoilage for thousands of years using many simple methods to determine whether food is safe (such as smell, feel, etc.) as well as more complex methods. One of the key requirements for food safety throughout the harvesting, preparation, and shipment process is temperature control. In addition, monitoring for specific indicators of spoilage (like ethane, ethanol, amines, or other volatile organic compounds (VOCs)) allows detection of spoilage before money is spent transporting food. Additionally, food sometimes spoils in route to consumers, and sometimes spoils while a consumer is storing it prior to preparation.

Thus, consumer demand for food safety continues to increase along with further improvements in food handling and processing technology. These improvements may be applicable to other perishable goods and associated technologies. Therefore, consumer safety could be increased, and money could be saved, by detecting spoilage earlier and/or more accurately during the transport and storage processes. Accordingly, there is a desire for simple and effective ways to monitor temperature profiles and detect spoilage during the food's travel from farm to plate.

### BRIEF SUMMARY

The systems, methods, and devices of the disclosure each have several aspects, no single one of which is solely responsible for its desirable attributes. Without limiting the scope of this disclosure as expressed by the claims which follow, some features will now be discussed briefly. After considering this discussion, and particularly after reading the section entitled “Detailed Description” one will understand how the features of this disclosure provide advantages that include improved communications between access points and stations in a wireless network.

Certain aspects provide a sensor apparatus for a perishable item. The sensor apparatus generally includes a lower frequency (LF) power receiver that is configured to receive power from a LF wireless transmission, a higher frequency (HF) power receiver that is configured to receive power from a HF wireless transmission, and a LF transmitter configured to transmit sensor data using the power received by the HF power receiver from the HF wireless transmission.

According to certain aspects, the LF transmitter is configured to transmit the sensor data concurrently with the HF power receiver receiving power from the HF wireless transmission and powering the LF transmitter.

According to certain aspects, a HF power transmitter may be configured to transmit the sensor data using power collected by the LF power receiver.

Certain aspects provide a method for powering a sensor apparatus. The method generally includes receiving, using at least one of a lower frequency (LF) power receiver or a higher frequency (HF) power receiver, power at the sensor apparatus from at least one of a LF wireless transmission or a HF wireless transmission, respectively, and powering, using the HF power receiver, a LF transmitter to transmit sensor data using the power received from the HF wireless transmission.

Certain aspects of the present disclosure provide an apparatus for sensing a perishable item. The apparatus generally includes first means for receiving power via a lower frequency (LF) wireless transmission; second means for receiving power via a higher frequency (HF) wireless transmission; means for sensing data; and means for transmitting an indication of the sensed data, the second means for receiving power being configured to power the means for transmitting using the power received from the HF wireless transmission.

Aspects generally include methods, apparatus, and systems, as substantially described herein with reference to and as illustrated by the accompanying drawings.

To the accomplishment of the foregoing and related ends, the one or more aspects comprise the features hereinafter fully described and particularly pointed out in the claims. The following description and the annexed drawings set forth in detail certain illustrative features of the one or more aspects. These features are indicative, however, of but a few of the various ways in which the principles of various aspects may be employed, and this description is intended to include all such aspects and their equivalents.

### BRIEF DESCRIPTION OF THE DRAWINGS

So that the manner in which the above-recited features of the present disclosure can be understood in detail, a more particular description, briefly summarized above, may be had by reference to aspects, some of which are illustrated in the appended drawings. It is to be noted, however, that the appended drawings illustrate only certain typical aspects of this disclosure and are therefore not to be considered limiting of its scope, for the description may admit to other equally effective aspects.

FIG. 1 is a block diagram illustrating an example sensor apparatus, in accordance with certain aspects of the present disclosure.

FIG. 2 illustrates example operations for powering a sensor apparatus, in accordance with certain aspects of the present disclosure.

FIG. 3 is a block diagram illustrating an example sensor apparatus, in accordance with certain aspects of the present disclosure.

FIG. 4 is a circuit diagram illustrating an example low frequency power receiver, in accordance with certain aspects of the present disclosure.

FIG. 5 is a circuit diagram illustrating an example high frequency power receiver, in accordance with certain aspects of the present disclosure.

FIG. 6 is a circuit diagram illustrating an example of combining outputs of receivers, in accordance with certain aspects of the present disclosure.

FIG. 7 is a circuit diagram illustrating an example low frequency receiver with two varieties of signaling transmitters, in accordance with certain aspects of the present disclosure.

To facilitate understanding, identical reference numerals have been used, where possible, to designate identical elements that are common to the figures. It is contemplated that elements disclosed in one aspect may be beneficially utilized on other aspects without specific recitation.

### DETAILED DESCRIPTION

Aspects of the present disclosure provide apparatus, methods, processing systems, and computer readable mediums for implementing a multi-power source perishable item sensor apparatus, such as a microwavable food sensor.

The following description provides examples, and is not limiting of the scope, applicability, or examples set forth in the claims. Changes may be made in the function and arrangement of elements discussed without departing from the scope of the disclosure. Various examples may omit, substitute, or add various procedures or components as appropriate. For instance, the methods described may be performed in an order different from that described, and various steps may be added, omitted, or combined. Also, features described with respect to some examples may be combined in some other examples. For example, an apparatus may be implemented or a method may be practiced using any number of the aspects set forth herein. In addition, the scope of the disclosure is intended to cover such an apparatus or method which is practiced using other structure, functionality, or structure and functionality in addition to or other than the various aspects of the disclosure set forth herein. It should be understood that any aspect of the disclosure disclosed herein may be embodied by one or more elements of a claim. The word “exemplary” is used herein to mean “serving as an example, instance, or illustration.” Any aspect described herein as “exemplary” is not necessarily to be construed as preferred or advantageous over other aspects.

### Examples of Multi-Power Source Perishable Item Sensor Apparatus

In accordance with one or more aspects disclosed herein, powering of small sensors inserted into food to allow temperature to be tracked and allow spoilage compounds to be detected may be provided. According to one or more cases, this may be accomplished with multi-mode wireless power supplies. Further, these power systems may also support communication via reverse impedance signaling systems.

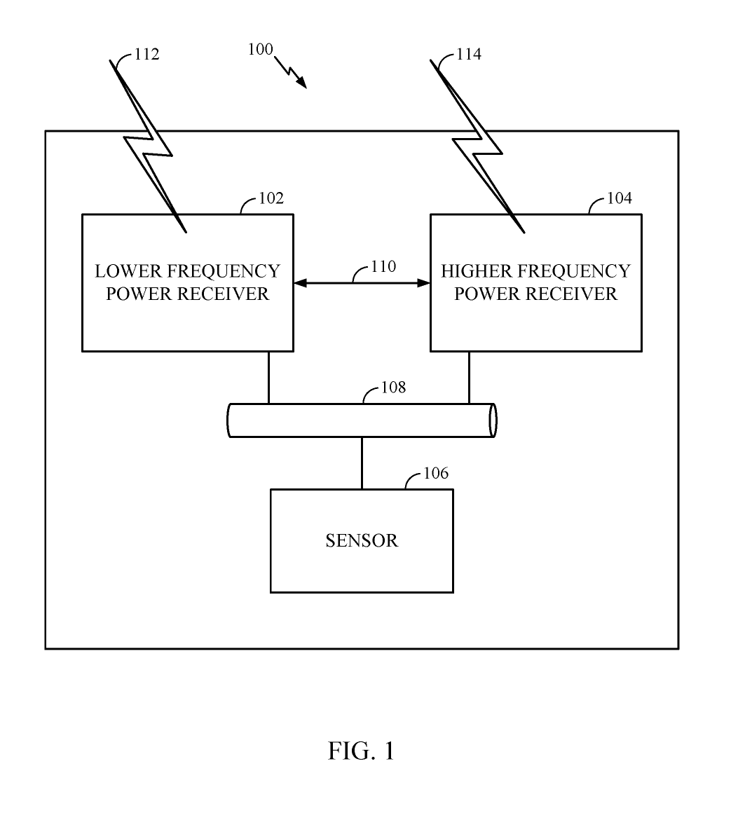

For example, FIG. 1 is a block diagram illustrating an example sensor apparatus 100, in accordance with certain aspects of the present disclosure. The sensor apparatus includes a lower frequency (LF) power receiver 102 that is configured to receive power from a LF wireless transmission 112. The sensor apparatus 100 also includes a higher frequency (HF) power receiver 104 that is configured to receive power from a HF wireless transmission 114. According to one or more cases, the HF power receiver 104 is configured to power the LF power receiver 102 such that the LF power receiver 102 functions as a LF transmitter to transmit sensor data from a sensor 106 using the power received from the HF wireless transmission 114. Sensor data may be transmitted using a wired connection 108 or may be wirelessly transmitted between elements. Additionally the wired connection 108 may also be configured to carry power between the LF power receiver 102, the HF power receiver 104, and the sensor 106. Further, according to another aspect, the power may be transmitted using a dedicated power connection 110 between the LF power receiver 102 and the HF power receiver 104. According to one or more cases, the power received by either of the LF power receiver 102 or the HF power receiver 104 may be stored in an energy storage device, such as a battery or a capacitor.

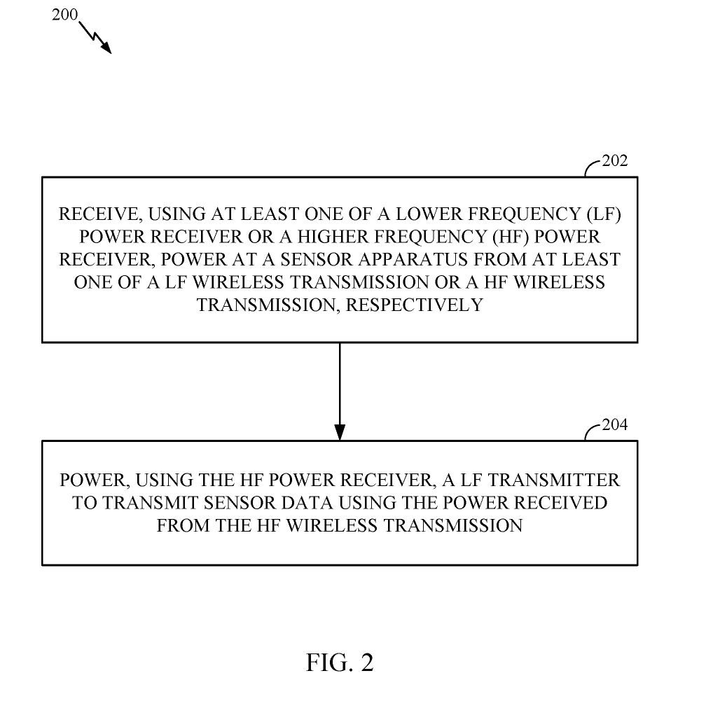

FIG. 2 illustrates example operations 200 for powering a sensor apparatus, in accordance with certain aspects of the present disclosure. Specifically, operations 200 begin, at block 202, with receiving, using at least one of a lower frequency (LF) power receiver or a higher frequency (HF) power receiver, power at the sensor apparatus from at least one of a LF wireless transmission or a HF wireless transmission, respectively. Further, operations 200 also include, at block 204, powering, using the HF power receiver, a LF transmitter to transmit sensor data using the power received from the HF wireless transmission.

In accordance with one or more cases, wirelessly powered food sensors, including sensors that are powered by microwave ovens may be provided. These sensors may be used to monitor food freshness, detect spoilage, and determine when a food item is finished cooking by collecting sensor data relating to a perishable item. According to one or more aspects, such a sensor may operate from either low frequency signals (e.g., standard wireless power signals or inductive cooking signals) or high frequency signals (e.g., from a microwave oven).

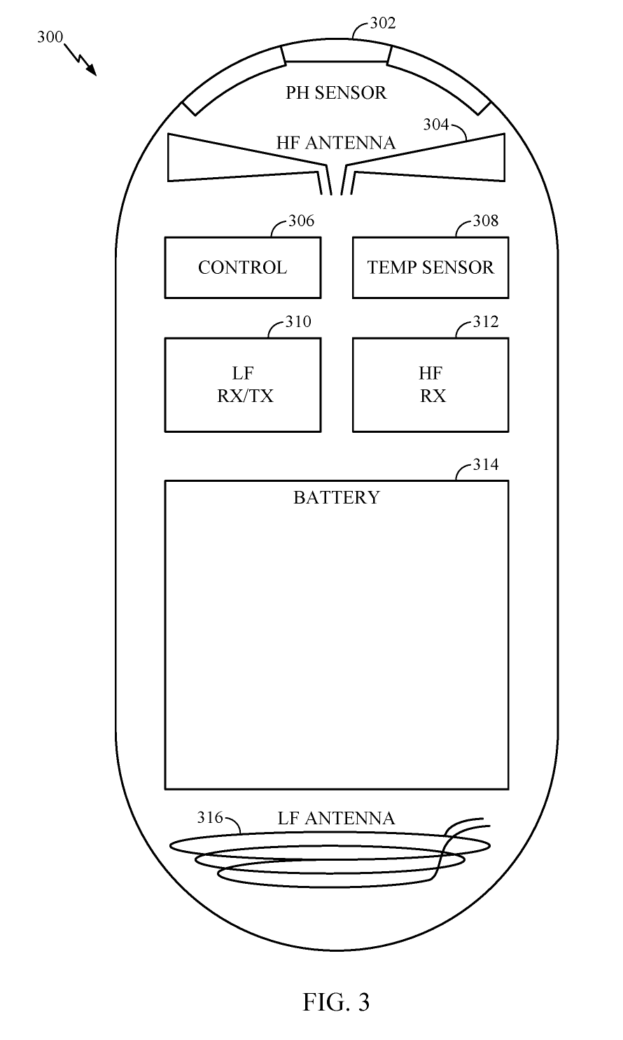

FIG. 3 is a block diagram illustrating an example sensor apparatus 300, in accordance with certain aspects of the present disclosure. Many food sensors may be designed to be very small. For example, the sensors may be the size of a turkey pop-up thermometer or smaller. The sensors may be inserted in the food shortly after harvesting or processing, and removed by the consumer before eating. The sensors may be designed to be small, smooth, and biologically inert so that accidental consumption is not dangerous.

As shown, the sensor apparatus 300 in FIG. 3 includes one or more sensors. For example, the sensor apparatus may include a food spoilage sensor configured to collect the sensor data relating to the perishable item. Specifically, the sensor apparatus 300 may include a pH sensor 302 and/or a temperature sensor 308. The sensor apparatus may also include a lower frequency (LF) transmitter and receiver 310 and a higher frequency (HF) receiver 312. The sensor apparatus 300 may include a LF antenna 316 that is connected to the LF transmitter and receiver 310. Further, the sensor apparatus 300 may include a HF antenna 304 that is connected to the HF receiver 312. The sensor apparatus 300 may also include a controller 306 and a battery 314 as shown. The battery 314 may be configured to store power collected from at least one of the LF transmitter and receiver 310 or the HF receiver 312.

According to one or more cases, the specific placement and number of sensors, controllers, batteries, antennas, and LF/HF receivers may vary. Further, according to one or more cases, the length of the sensor apparatus 300 of FIG. 3 may be approximately 10 mm. In other cases, the length may be smaller or larger depending on the number, placement, and size of elements included. For example, in one case, the battery may be smaller and/or a number of additional sensors may be included. Additionally, in accordance with one or more examples, the dimensions of the sensor apparatus can vary from that which is shown in FIG. 3.

During operation one or more sensors may be designed to detect spoilage indicators such as pH, amine levels, temperature, ethanol levels, ammonia levels, and/or ethane levels. The sensor(s) may further log those indicators into a small local storage device. This may be done at a low duty cycle (for example, one reading every three hours) thus allowing for days of operation on a very small internal battery. In accordance with other aspects, the duty cycle may be shorter (for example, a reading every few minutes), or longer (for example, daily or weekly), depending on the perishable item, sensor capabilities, and monitoring requirements.

The sensor data can be transmitted via several means, including through a low-to-medium-frequency data transmitter. However, these low-to-medium-frequency data transmitters may consume more power to operate as compared to the sensors. Further, because the data may need to be made available to a potential consumer between being taken out of storage and consumed, the data may be transmitted more frequently. For example, one transmission every three hours would be insufficient for a steak that could go from refrigerator to plate in 20 minutes.

Thus, a small sensor with its resulting small battery may have constraints limiting the sensor's ability to transmit its data regularly with a high enough frequency for a consumer to receive the information in a timely fashion. According to one or more cases, this can be overcome with a radio frequency identifier (RFID)-like scheme, where an external signal provides power wirelessly to the radio portion of the sensor.

There are one or more considerations regarding the electrical design of such a power receiver that may limit a sensor device, which may be addressed as follows. For example, the small size of the sensor leads to some challenges with wireless power transmission. Small antennas are suited for receiving high frequency power, because the antenna can be “electrically large” compared to the wavelength. However, high frequencies do not penetrate moist and/or conductive materials very well, and so lower frequencies are often used for power transmission in, for example, medically implanted devices.

The high frequency limitation can be overcome by transmitting at very high powers. These powers are often seen in microwave ovens, and microwave ovens are commonly used to prepare packaged food and heat food shortly before consumption. This availability of high power, high frequency (e.g., RF) means that the sensor can have a high frequency (2.4 GHz) antenna and still get enough signal strength through the food to power the sensor. This is useful, since final food preparation in a microwave is an excellent place to obtain a final data dump from the sensor to verify that the food is safe to eat. Accordingly, in one or more cases, the HF power receiver may be configured to receive the power from a microwave device transmission.

The low frequency limitation (inefficient receive antennas) can likewise be overcome with high field strengths. At low frequencies, RF penetrates food easily, but an inefficient antenna may need a strong signal to produce enough power to overcome losses (like rectification losses inside the device) and produce power for the sensor. This lower frequency RF (e.g., in the range of tens of kHz to tens of MHz) can be provided by several cases. One case includes, for example, a strong emitter intended to generate a strong enough field for purposes of powering the sensor. Another case includes an induction cooker that may generate strong low frequency fields during normal operation. Again this is useful, since final food preparation on an induction cooker is an excellent place to obtain a final data dump from the sensor to verify that the food is safe to eat.

Thus, in accordance with one or more cases, the sensor apparatus may have a dual frequency design. Specifically, the sensor apparatus may be designed to be able to receive power at both a low and a high frequency, thus maximizing ability to read out the data under a variety of conditions of storage and preparation. According to one or more cases, an example choice for frequencies would be 2.4 GHz for high frequency operation and 6.78 MHz for low frequency operation.

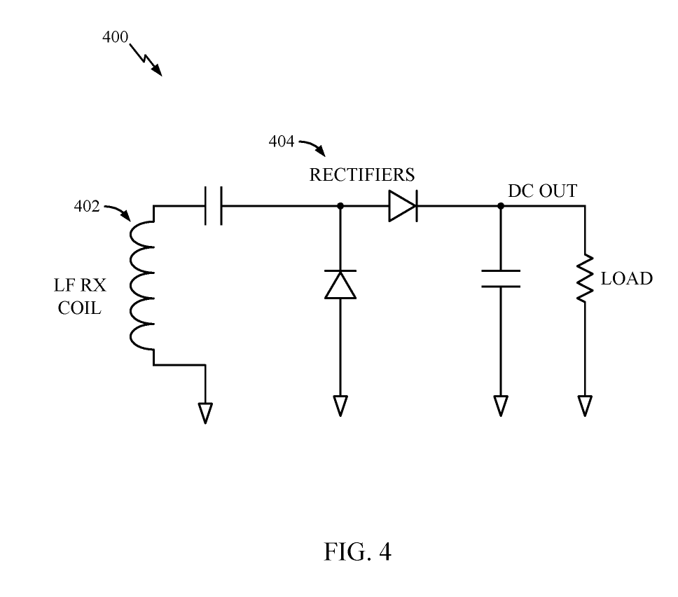

An example of a power receiver is shown in FIG. 4. Specifically, FIG. 4 depicts a circuit diagram illustrating an example low frequency power receiver 400, in accordance with certain aspects of the present disclosure. The low frequency power receiver 400 contains a resonant receive coil 402 coupled to a half-wave rectifier circuit 404 via a coupling capacitor. A bypass or filter capacitor may be coupled to the output of the half-wave rectifier circuit 404 to filter out or at least reduce ripple and other high frequency content in the load.

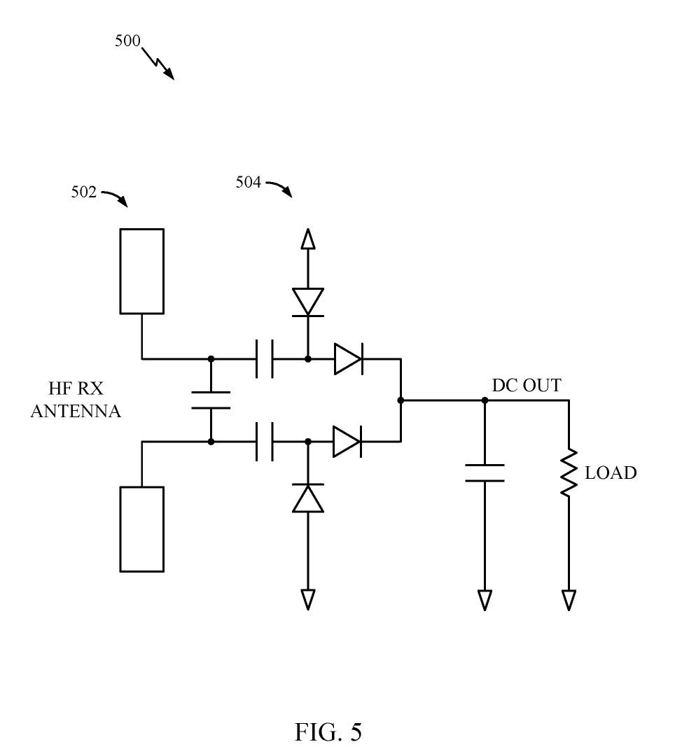

FIG. 5 is a circuit diagram illustrating an example high frequency power receiver 500, in accordance with certain aspects of the present disclosure. The high frequency power receiver 500 contains a rectenna-style power receiver 502 with a tuning circuit and a set of rectifiers 504.

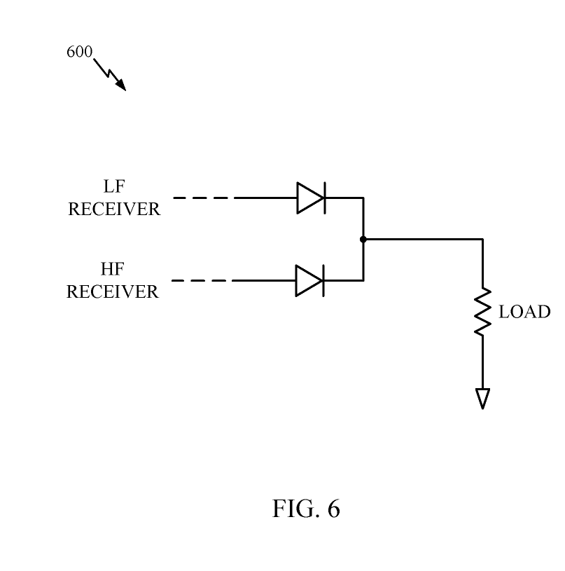

According to one or more cases, the outputs of a LF power receiver and HF power receiver may be connected together. The outputs can either be connected directly together (e.g., the loads shown in FIGS. 4 and 5 can be the same load), or the outputs can be diode-ORed as shown in FIG. 6. Specifically, FIG. 6 is a circuit diagram 600 illustrating an example of combining outputs of the LF and HF receivers, in accordance with certain aspects of the present disclosure.

In one or more cases, low frequencies (in the range of tens of kHz to tens of MHz) may be selected for transmitting the sensor data. This may be accomplished in a number of different ways. For example, according to one case, during high frequency operation, the high frequency receiver may receive power from a high frequency signal. The high frequency receiver may then provide power to the LF power receiver and transmitter. The LF power transmitter may then operate using the provided power in a “backwards” manner by sending a low frequency data signal that contains the sensor data from the power reception antenna associated with the LF power receiver. A well-designed power reception antenna may, by design, be a good transmitter at the same frequency. For example, in accordance with one or more cases, a microwave oven may send out a signal at 2.4 GHz that is received by a HF power receiver of a sensor apparatus. The sensor apparatus may then transmit the sensor data at 6.78 MHz using, for example, a LF power receiver antenna as a transmitter. A receive antenna inside the oven may then receive the sensor data.

During low frequency operation, the low frequency signal can provide power for the sensor, and the sensor may then vary its impedance to send a signal back to the power transmitter. This feature may be implemented using common RFID standards, such as IEC 14443 and IEC 18000.

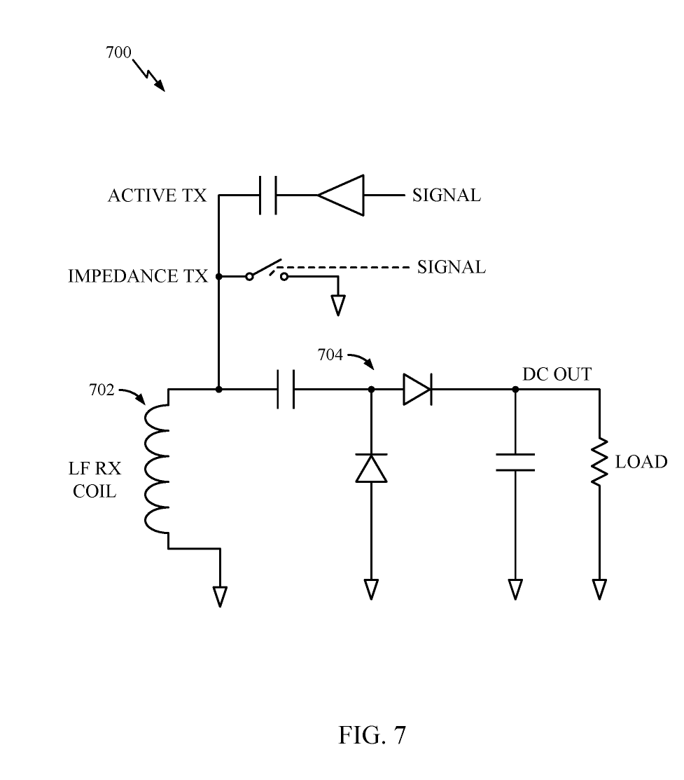

FIG. 7 is a circuit diagram illustrating an example low frequency transceiver 700 with two varieties of signaling transmitters, in accordance with certain aspects of the present disclosure. Specifically, the circuit diagram of FIG. 7 shows a low frequency power transceiver that can utilize both impedance signaling and transmissive signaling. Specifically, the LF transceiver 700 includes a resonant coil 702 connected to a rectifier 704 similar to the LF receiver of FIG. 4. Additionally, the LF transceiver 700 includes an active transmitter (Tx) and an impedance Tx as shown that provide the impedance signaling and transmissive signaling, respectively. According to one or more examples, a transmit signal may come from the controller and represent the desired modulation scheme.

According to one or more cases, the sensor apparatus may directly provide an indication to a user of the food safety information derived from the collected sensor data (with or without transmission of any sensor data). For example, a light-emitting diode (LED) of the sensor apparatus could light up to indicate that the food was safe (or not safe) for consumption based on the sensor data. As another example, a speaker may emit a chirp or other audio signal as an indication of food safety.

In accordance with one or more cases, data transmitted from the sensor apparatus may be received and interpreted to provide food safety information to a consumer. For example, once the data is transmitted to a data receive antenna, a controller may receive the data and make a determination on whether the food is safe to eat. The controller may use an adjustable threshold, so that food for an infant is tested to a higher standard than food for an adult. The controller may use other information provided by the sensor apparatus, like encoded information on the type of food, to determine which set of standards to use. The controller may use information obtained from the Internet, like weather information between the farm where the food was grown and the place of consumption, to modify its standards. For example, hot weather may make food more susceptible to spoilage and may be useful to know if the sensor apparatus does not contain a temperature sensor.

The information based on the sensor data may be provided to a user in a number of different ways including visual, auditory (e.g., sound), tactile (e.g., vibration), and/or a combination thereof. For example, the output may be either a simple warning (a buzzer or display that indicates the food is unsafe) or a more complex display, like the odds the food is safe, the percent estimated spoilage of the food, or the remaining time the food will be safe to eat at its current temperature.

In some cases the controller may use a separate wireless connection to connect to an external alarm system, an external smartphone, and/or a monitoring application within the cloud (e.g., connecting via IEEE 802.11). In some cases the controller itself may be a smartphone or other portable device. Thus the sensor may communicate directly with the smartphone without a separate controller.

According to one or more cases, several ways may be provided for reading out data, all of which may be supported by the sensor apparatus system. For example, a LF receiver coil near/in a microwave oven or other microwave emitter may be provided for receiving the sensor data from the sensor apparatus. Another example includes an induction cooker with an integrated LF data receiver coil. In another case, a standalone LF emitter/receiver (for example, an RFID reader) may be provided. Further, other ways to accomplish the sensor data reception may also be implemented.

The sensor apparatus may be used in conjunction with other food safety methods. Additionally the sensor apparatus may have some utility in specific applications including but not limited to sealed containers of food to help provide high-confidence assurance of food safety and for liability purposes to determine cause of spoilage.

According to one or more cases, the sensor apparatus may be inserted in food and can be seen by X-raying food or by chopping up the product and looking for the device. The sensor apparatus may also provide features of a “shipping tracker” applied to food while also providing the combination of a LF and HF receiver to allow powering from a wide variety of cooking methods.

The methods disclosed herein comprise one or more steps or actions for achieving the described method. The method steps and/or actions may be interchanged with one another without departing from the scope of the claims. In other words, unless a specific order of steps or actions is specified, the order and/or use of specific steps and/or actions may be modified without departing from the scope of the claims.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c). As used herein, including in the claims, the term “and/or,” when used in a list of two or more items, means that any one of the listed items can be employed by itself, or any combination of two or more of the listed items can be employed. For example, if a composition is described as containing components A, B, and/or C, the composition can contain A alone; B alone; C alone; A and B in combination; A and C in combination; B and C in combination; or A, B, and C in combination.

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” For example, the articles “a” and “an” as used in this application and the appended claims should generally be construed to mean “one or more” unless specified otherwise or clear from the context to be directed to a singular form. Unless specifically stated otherwise, the term “some” refers to one or more. Moreover, the term “or” is intended to mean an inclusive “or” rather than an exclusive “or.” That is, unless specified otherwise, or clear from the context, the phrase, for example, “X employs A or B” is intended to mean any of the natural inclusive permutations. That is, for example the phrase “X employs A or B” is satisfied by any of the following instances: X employs A; X employs B; or X employs both A and B. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.”

The various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

The various illustrative logical blocks, modules and circuits described in connection with the present disclosure may be implemented or performed with a general purpose processor, a digital signal processor (DSP), an application specific integrated circuit (ASIC), a field programmable gate array (FPGA) or other programmable logic device (PLD), discrete gate or transistor logic, discrete hardware components, or any combination thereof designed to perform the functions described herein. A general-purpose processor may be a microprocessor, but in the alternative, the processor may be any commercially available processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

If implemented in hardware, an example hardware configuration may comprise a processing system in a wireless node. The processing system may be implemented with a bus architecture. The bus may include any number of interconnecting buses and bridges depending on the specific application of the processing system and the overall design constraints. The bus may link together various circuits including a processor, machine-readable media, and a bus interface. The bus interface may be used to connect a network adapter, among other things, to the processing system via the bus. The network adapter may be used to implement the signal processing functions of the physical (PHY) layer. The bus may also link various other circuits such as timing sources, peripherals, voltage regulators, power management circuits, and the like, which are well known in the art, and therefore, will not be described any further. The processor may be implemented with one or more general-purpose and/or special-purpose processors. Examples include microprocessors, microcontrollers, DSP processors, and other circuitry that can execute software. Those skilled in the art will recognize how best to implement the described functionality for the processing system depending on the particular application and the overall design constraints imposed on the overall system.

If implemented in software, the functions may be stored or transmitted over as one or more instructions or code on a computer readable medium. Software shall be construed broadly to mean instructions, data, or any combination thereof, whether referred to as software, firmware, middleware, microcode, hardware description language, or otherwise. Computer-readable media include both computer storage media and communication media including any medium that facilitates transfer of a computer program from one place to another. The processor may be responsible for managing the bus and general processing, including the execution of software modules stored on the machine-readable storage media. A computer-readable storage medium may be coupled to a processor such that the processor can read information from, and write information to, the storage medium. In the alternative, the storage medium may be integral to the processor. By way of example, the machine-readable media may include a transmission line, a carrier wave modulated by data, and/or a computer readable storage medium with instructions stored thereon separate from the wireless node, all of which may be accessed by the processor through the bus interface. Alternatively, or in addition, the machine-readable media, or any portion thereof, may be integrated into the processor, such as the case may be with cache and/or general register files. Examples of machine-readable storage media may include, by way of example, RAM (Random Access Memory), flash memory, phase change memory, ROM (Read Only Memory), PROM (Programmable Read-Only Memory), EPROM (Erasable Programmable Read-Only Memory), EEPROM (Electrically Erasable Programmable Read-Only Memory), registers, magnetic disks, optical disks, hard drives, or any other suitable storage medium, or any combination thereof. The machine-readable media may be embodied in a computer-program product.

A software module may comprise a single instruction, or many instructions, and may be distributed over several different code segments, among different programs, and across multiple storage media. The computer-readable media may comprise a number of software modules. The software modules include instructions that, when executed by an apparatus such as a processor, cause the processing system to perform various functions. The software modules may include a transmission module and a receiving module. Each software module may reside in a single storage device or be distributed across multiple storage devices. By way of example, a software module may be loaded into RAM from a hard drive when a triggering event occurs. During execution of the software module, the processor may load some of the instructions into cache to increase access speed. One or more cache lines may then be loaded into a general register file for execution by the processor. When referring to the functionality of a software module below, it will be understood that such functionality is implemented by the processor when executing instructions from that software module.

Also, any connection is properly termed a computer-readable medium. For example, if the software is transmitted from a website, server, or other remote source using a coaxial cable, fiber optic cable, twisted pair, digital subscriber line (DSL), or wireless technologies such as infrared (IR), radio, and microwave, then the coaxial cable, fiber optic cable, twisted pair, DSL, or wireless technologies such as infrared, radio, and microwave are included in the definition of medium. Disk and disc, as used herein, include compact disc (CD), laser disc, optical disc, digital versatile disc (DVD), floppy disk, and Blu-ray® disc where disks usually reproduce data magnetically, while discs reproduce data optically with lasers. Thus, in some aspects computer-readable media may comprise non-transitory computer-readable media (e.g., tangible media). In addition, for other aspects computer-readable media may comprise transitory computer-readable media (e.g., a signal). Combinations of the above should also be included within the scope of computer-readable media.

Thus, certain aspects may comprise a computer program product for performing the operations presented herein. For example, such a computer program product may comprise a computer-readable medium having instructions stored (and/or encoded) thereon, the instructions being executable by one or more processors to perform the operations described herein. For example, instructions for performing the operations described herein and illustrated in FIG. 2.

Further, it should be appreciated that modules and/or other appropriate means for performing the methods and techniques described herein can be downloaded and/or otherwise obtained by a user terminal and/or base station as applicable. For example, such a device can be coupled to a server to facilitate the transfer of means for performing the methods described herein. Alternatively, various methods described herein can be provided via storage means (e.g., RAM, ROM, a physical storage medium such as a compact disc (CD) or floppy disk, etc.), such that a user terminal and/or base station can obtain the various methods upon coupling or providing the storage means to the device. Moreover, any other suitable technique for providing the methods and techniques described herein to a device can be utilized.

It is to be understood that the claims are not limited to the precise configuration and components illustrated above. Various modifications, changes and variations may be made in the arrangement, operation and details of the methods and apparatus described above without departing from the scope of the claims.

1. A sensor apparatus for a perishable item, the sensor apparatus comprising:
a lower frequency (LF) power receiver that is configured to receive power from a LF wireless transmission;
a higher frequency (HF) power receiver that is configured to receive power from a HF wireless transmission; and
a LF transmitter configured to transmit sensor data using the power received by the HF power receiver from the HF wireless transmission.

2. The sensor apparatus of claim 1, further comprising:
at least one food spoilage sensor configured to collect the sensor data relating to the perishable item.

3. The sensor apparatus of claim 2, wherein the at least one food spoilage sensor is powered by the power received by at least one of the LF power receiver or the HF power receiver.

4. The sensor apparatus of claim 2, wherein the at least one food spoilage sensor comprises at least one of a temperature sensor or a pH sensor.

5. The sensor apparatus of claim 1, wherein the LF transmitter is configured to transmit the sensor data concurrently with the HF power receiver receiving power from the HF wireless transmission and powering the LF transmitter.

6. The sensor apparatus of claim 1, further comprising:
a HF power transmitter configured to transmit the sensor data using power collected by the LF power receiver.

7. The sensor apparatus of claim 1, further comprising:
a battery that is configured to store power collected from at least one of the LF power receiver or the HF power receiver;
a LF antenna configured to at least one of receive the LF wireless transmission or transmit the sensor data; and
a HF antenna configured to receive the HF wireless transmission.

8. The sensor apparatus of claim 1,
wherein the LF power receiver comprises a resonant coil coupled to a rectifier circuit.

9. The sensor apparatus of claim 1,
wherein the HF power receiver comprises a rectenna-style power receiver that includes a tuning circuit and a set of rectifiers.

10. The sensor apparatus of claim 1,
wherein the LF power receiver is configured to receive the power from an induction device transmission, and
wherein the HF power receiver is configured to receive the power from a microwave device transmission.

11. The sensor apparatus of claim 1, wherein the sensor apparatus is configured to detect one or more spoilage indicators of the perishable item.

12. The sensor apparatus of claim 1,
wherein the HF power receiver is configured for the HF wireless transmission at 2.4 GHz, and
wherein the LF transmitter is configured to transmit the sensor data at 6.78 MHz.

13. A method for powering a sensor apparatus, the method comprising:
receiving, using at least one of a lower frequency (LF) power receiver or a higher frequency (HF) power receiver, power at the sensor apparatus from at least one of a LF wireless transmission or a HF wireless transmission, respectively; and
powering, using the HF power receiver, a LF transmitter to transmit sensor data using the power received from the HF wireless transmission.

14. The method of claim 13, further comprising:
collecting, using at least one food spoilage sensor, the sensor data relating to the perishable item.

15. The method of claim 14, wherein the at least one food spoilage sensor is powered by the power received by at least one of the LF power receiver or the HF power receiver.

16. The method of claim 14, wherein the at least one food spoilage sensor comprises at least one of a temperature sensor or a pH sensor.

17. The method of claim 13, further comprising:
transmitting the sensor data using the LF transmitter concurrently with the HF power receiver receiving power from the HF wireless transmission and powering the LF transmitter.

18. The method of claim 13, further comprising:
transmitting, using a HF power transmitter, the sensor data using power collected by the LF power receiver.

19. The method of claim 13, further comprising:
storing power collected from at least one of the LF power receiver or the HF power receiver in a battery;
receiving the LF wireless transmission or transmitting the sensor data using a LF antenna; and
receiving the HF wireless transmission using a HF antenna.

20. The method of claim 13,
wherein the LF power receiver comprises a resonant coil coupled to a rectifier circuit.

21. The method of claim 13,
wherein the HF power receiver comprises a rectenna-style power receiver that includes a tuning circuit and a set of rectifiers.

22. The method of claim 13, further comprising
receiving, at the LF power receiver, the power from an induction device transmission, and
receiving, at the HF power receiver, the power from a microwave device transmission.

23. The method of claim 13, further comprising:
detecting one or more spoilage indicators of the perishable item.

24. The method of claim 13,
wherein the HF power receiver is configured for the HF wireless transmission at 2.4 GHz, and
wherein the LF transmitter is configured to transmit the sensor data at 6.78 MHz.

25. An apparatus for sensing a perishable item, comprising:
first means for receiving power via a lower frequency (LF) wireless transmission;
second means for receiving power via a higher frequency (HF) wireless transmission;
means for sensing data; and
means for transmitting an indication of the sensed data, the second means for receiving power being configured to power the means for transmitting using the power received from the HF wireless transmission.
